SEQUENCE LISTING (17 sequences)

SEQ ID NO: 1 - protein, 102 aa, Artificial Sequence
  Consensus Sequence
XXEWXXXXCXXRXXXXXXXXXXCXXXXXXXXXXXXXXXXXXXXXXXXXXXXXXCXXXCXXYXXXXXXXXXXXXXXXXXXXXXXXXXXXXXXXXXXXXXXXXX

SEQ ID NO: 2 - protein, 67 aa, Plasmodium falciparum
LTEWYDDYCYTRQKYLKDVQEKCKSNDQLKCDTECNKKCEDYEKYMKKKKEWIPQDKYYKDERDKKR

SEQ ID NO: 3 - protein, 85 aa, Plasmodium falciparum
LQEWVEHFCKQRQEKVKPVIENCKSCKESGGTCNGECKTECKNKCEVYKKFIEDCKGGDGTAGSSWVKRWDQIYKRYSKYIEDAK

SEQ ID NO: 4 - protein, 88 aa, Plasmodium falciparum
LQEWVENFCEQRQAKVKDVITNCKSCKESGNKCKTECKTKCKDECEKYKKFIEACGTAGGGIGTAGSPWSKRWDQIYKRYSKHIEDAK

SEQ ID NO: 5 - protein, 68 aa, Plasmodium falciparum
ITEWYDDYCHTRQKYLKDVKEKCKSNDQLKCDKECNNKCDEYKKYMEGKKKEWDAQYKYYKEQRNKKE

SEQ ID NO: 6 - protein, 70 aa, Plasmodium falciparum
FVEWYDDYCKERQKYLTEVASTCKSIDGGQLKCDRGCNNKCDEYKKYMRKKKEEWNLQDKYYKDKRENKG

SEQ ID NO: 7 - protein, 74 aa, Plasmodium falciparum
YVEWSDEFCRERKKLEDKVEDVCIKAKDYEGCKNNKSNNSCVKVCKEYENYITGKKTQYESQEGKFNTEKRQKK

SEQ ID NO: 8 - protein, 81 aa, Plasmodium falciparum
FKEWGEQFCIERLRYEQNIREACTINGKNEKKCINSKSGQGDKIQGACKRKCEKYKKYISEKKQEWDKQKTKYENKYVGKS

SEQ ID NO: 9 - protein, 84 aa, Plasmodium falciparum
FTEWSDEFCTERSIKIKELETKCNDCTVSESGTSDATGNKTCDDKDKCDECKRACTTYKTWLKNWKTQYKTQSKKYFDDKRKEL

SEQ ID NO: 10 - protein, 80 aa, Plasmodium falciparum
FTEWGEDFCKNRKKELVSLKKKCDSCTLRNNGTSNKTCDDNENCGACKTQCEKYKKWMERWKKHYSSQKKKFQLYKNSAT

SEQ ID NO: 11 - protein, 73 aa, Plasmodium falciparum
FQEWTENFCTKRNELYENMVTACNSAKCNTSNGSVDKKECTEACKNYSNFILIKKKEYQSLNSQYDMNYKETK

SEQ ID NO: 12 - protein, 312 aa, Plasmodium falciparum
KCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTK

SEQ ID NO: 13 - protein, 317 aa, Plasmodium falciparum
KCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKN

SEQ ID NO: 14 - protein, 322 aa, Plasmodium falciparum
DLNATNYIRGCQSKTYDGKIFPGKGGEKQWICKDTIIHGDTNGACIPPRTQNLCVGELWDKSYGGRSNIKNDTKELLKEKIKNAIHKETELLYEYHDTGTAIISKNDKKGQKGKNDPNGLPKGFCHAVQRSFIDYKNMILGTSVNIYEHIGKLQEDIKKIIEKGTPQQKDKIGGVGSSTENVNAWWKGIEREMWDAVRCAITKINKKNNNSIFNGDECGVSPPTGNDEDQSVSWFKEWGEQFCIERLRYEQNIREACTINGKNEKKCINSKSGQGDKIQGACKRKCEKYKKYISEKKQEWDKQKTKYENKYVGKSASDLLKE

SEQ ID NO: 15 - protein, 272 aa, Plasmodium falciparum
IYRLKHHEYDKGNDYICNKYKNINVNMKKNNDDTWTDLVKNSSDINKGVLLPPRRKNLFLKIDESDICKYKRDPKLFKDFIYSSAISEVERLKKVYGEAKTKVVHAMKYSFADIGSIIKGDDMMENNSSDKIGKILGDGVGQNEKRKKWWDMNKYHIWESMLCGYKHAYGNISENDRKMLDIPNNDDEHQFLRWFQEWTENFCTKRNELYENMVTACNSAKCNTSNGSVDKKECTEACKNYSNFILIKKKEYQSLNSQYDMNYKETKAEKKE

SEQ ID NO: 16 - protein, 322 aa, Plasmodium falciparum; identical to SEQ ID NO: 14

SEQ ID NO: 17 - protein, 311 aa, Plasmodium falciparum
SEPIYIRGCQPKIYDGKIFPGKGGEKQWICKDTIIHGDTNGACIPPRTQNLCVGELWDKRYGGRSNIKNDTKESLKQKIKNAIQKETELLYEYHDKGTAIISRNPMKGQKEKEEKNNDSNGLPEGFCHAVQRSFIDYKDMILGTSVNIYEYIGKLQEDIKKIIEKGTTKQNGKTVGSGAENVNAWWKGIEGEMWDAVRCAITKINKKQKKNGTFSIDECGIFPPTGNDEDQSVSWFKEWSEQFCIERLQYEKNIRDACTNNGQGDKIQGDCKRKCEEYKKYISEKKQEWDKQKTKYENKYVGKSASDLLKE

(19) United States
(12) Patent Application Publication
(10) Pub. No.: US 2009/0130136 A1
(43) Pub. Date: May 21, 2009

(54) Chondroitin Sulphate a Binding Domains

(76) Inventors: Louis H. Miller, Cabin John, MD (US); Benoit Gamain, Saint Cyr L'Ecole (FR); Joseph D. Smith, Seattle, WA (US); Adama R. Trimnell, Seattle, WA (US); Christine Scheidig, Blennes (FR); Artur Scherf, Paris (FR)

Correspondence Address: KNOBBE, MARTENS, OLSON & BEAR, LLP, 2040 MAIN STREET, FOURTEENTH FLOOR, IRVINE, CA, 92614, US

(21) Appl. No.: 11/664,376

(22) PCT Filed: Sep. 30, 2005
(86) PCT No.: PCT/US05/35486

§ 371 Date: May 9, 2008

Related U.S. Application Data
(60) Provisional application No. 60/615,300, filed on Sep. 30, 2004.
(60) Provisional application No. 60/630,752, filed on Nov. 24, 2004.

(51) Int. Cl. A61K 39/002; C07K 14/445; C07H 21/00; C12N 15/63; A61P 33/06; C12P 21/00; C12N 1/00

(52) U.S. Cl. 424/192.1; 530/324; 536/23.7; 435/320.1; 435/69.1; 435/243; 424/272.1; 530/350

(57) ABSTRACT

The invention is related to the identification of CSA binding domains in var2CSA homologs from different parasite strains and furthermore to an isolated polypeptide comprising a CSA-binding domain sequence substantially as shown in SEQ ID NO:1, or functional equivalent thereof, or the corresponding portion of PfEMP1 from a strain of Plasmodium, substantially in isolation from sequences naturally occurring adjacent thereto in the PfEMP1 protein, and related nucleotide sequences, vectors, host cells, vaccines, and methods of use.

### RELATED APPLICATIONS

This application claims the benefit of priority of U.S. Provisional Application No. 60/615,300, filed Sep. 30, 2004, and U.S. Provisional Application No. 60/630,752, filed Nov. 24, 2004, both of which are hereby expressly incorporated by reference in their entireties.

### BACKGROUND OF THE INVENTION

Plasmodium falciparum infection during pregnancy is associated with parasitized erythrocyte (PE) sequestration in the placenta, and contributes to low birthweight babies and neonatal mortality (Brabin B. J. et al. 2004 Placenta 25:359-378). Placental isolates are functionally distinct because they do not bind CD36, but instead bind chondroitin sulphate A (CSA) (Fried M. & Duffy P. E. 1996 Science 272:1502-1504) and hyaluronic acid (Beeson J. G. et al. 2000 Nat Med 6:86-90). During pregnancy, antibodies develop to the PE surface, which are broadly, strain-transcendent and have been suggested to have a role in protective immunity (Fried M. et al. 1998 Nature 395:851-852).

P. falciparum-infected erythrocytes employ a variable family of erythrocyte surface adhesion ligands called var genes that encode P. falciparum erytirocyte membrane protein 1 (PfEMP1) (Miller L. H. et al. 2002 Nature 415:673-679) to sequester in different microvasculature sites. PfEMP1 proteins have multiple adhesion domains, called Duffy binding like (DBL) domains and cysteine-rich interdomain region (CIDR), which determine PE binding specificity (Miller L. H. et al. 2002 Nature 415:673-679). Of importance to placental sequestration, CD36 and CSA are mutually exclusive PE adhesion traits (Fried M. & Duffy P. E. 1996 Science 272:1502-1504; Rogerson S. J. et al. 1995 J Exp Med 182:15-20) that are functionally incompatible in the same PfEMP1 protein (Gamain B. et al. 2002 PNAS USA 99:10020-10024). Mechanistically, this may have arisen because CD36 binding and non-binding var genes localize to distinct chromosomal regions and are transcribed in opposite orientations (Robinson B. A. et al. 2003 Mol Microbiol 47:1265-1278; Lavstsen T. et al. 2003 Malar J 2:27; Kraemer S. M. et al. 2003 Mol Microbiol 50:1527-1538). This genetic organization may cause them to recombine separately and evolve different structures and functions under distinct selective pressures. As placental isolates do not bind CD36 (Fried M. & Duffy P. E. 1996 Science 272:1502-1504; Beeson J. G. et al. 2000 Nat Med 6:86-90), the maternal placenta selects for non-CD36 binding PfEMP1 proteins that ensure localization in placenta, not on vascular endothelium.

Within the group of PfEMP1 proteins predicted not to bind CD36, a small subset, including the var1CSA, have DBL-γ domains that bind CSA (Gamain B. et al. 2002 PNAS USA 99:10020-10024; Buffet P. A. et al. 1999 PNAS USA 96:12743-12748; Gamain B. et al. 2004 Mol Microbiol 53:445-455). Another gene within this group, var2CSA, does not contain DBL-γ domains but was recently found to be upregulated in PE that bind CSA (Salanti A. et al. 2003 Mol Microbiol 49:179-191). Var2CSA is considered an additional candidate for CSA-binding and placental adhesion because of its expression profile and lack of a typical CD36 binding domain. Of interest, the var2CSA contains sequence homology to the minimal CSA binding region from the var1CSA (Gamain B. et al. 2004 Mol Microbiol 53:445-455) and is unusually conserved between parasite strains (Kraemer S. M. et al. 2003 Mol Microbiol 50:1527-1538; Salanti A. et al. 2003 Mol Microbiol 49:179-191).

### SEGUE TO THE INVENTION

The aim of this study was to determine whether var2CSA contains CSA-binding domains.

### SUMMARY OF THE INVENTION

The invention is related to the identification of CSA binding domains in var2CSA homologs from different parasite strains and furthermore to an isolated polypeptide comprising a CSA-binding domain sequence substantially as shown in SEQ ID NO: 1, or functional equivalent thereof, or the corresponding portion of PfEMP1 from a strain of Plasmodium, substantially in isolation from sequences naturally occurring adjacent thereto in the PfEMP1 protein, and related nucleotide sequences, vectors, host cells, vaccines, and methods of use.

### BRIEF DESCRIPTION OF THE DRAWINGS

FIG. 1. Northern Blot analysis of var1CSA genetically disrupted parasites before (C1) and after reselection on CSA (C1-CSA). Total RNA from ring (R) and trophozoite (T) stages were size fractionated and hybridized with a probe specific for var2CSA DBL3-x domain (left) or the semi-conserved var exon II (right). The exon 2 probe labels full-sized var transcripts and smaller, approximately 3 kb “sterile” exon 2 transcripts that have been previously described. An RNA size standard is indicated in kilobases (kb). Autoradiogram was developed after 4 h.

FIG. 2. Comparison of CSA binding and non-binding domains from the var2CSA. (A) Alignment of the A4 (SEQ ID NO: 16) and 3D7 DBL3-X (SEQ ID NO: 17) expression constructs. The A4 sequence bound CSA, while the 3D7 sequence did not. The region corresponding to the minimal binding region from the FCR3 var1CSA DBL-γ minimal binding domain is highlighted in gray. Conserved cysteines present in that region are in bold and numbered by reference to the 5 cysteines present in the FCR3 var1CSA DBL-γ binding domain. Multiple sequence alignments were obtained using ClustalW software available at http://www.ebi.ac.uk/clustalw/. Symbols: *, identical or conserved residues; :, conserved substitution; ., semiconserved substitutions. (B) Alignment of the FCR3 var1CSA DBL-Y minimal binding domain with the corresponding regions from DBL-γ type (Gamain B. et al. 2004 Mol Microbiol 53:445-455) and DBL2X, DBL3-X, and DBL-ε in var2CSA domains (this study) that bind CSA. Cysteines are highlighted in gray and numbered from C1 to C5, based upon the var1CSA DBL3-γ minimal binding domain. The code for the consensus residues is h (hydrophobic), a (aromatic), − (negative charge), 1 (aliphatic), and a capital letter for a conserved amino acid in the single letter code.

FIG. 3. Alignment of the FCR3 var1CSA DBL3-γ minimal binding domain with the corresponding regions from other DBL domains that bound CSA. Cysteines are highlighted in gray and numbered from C1 to C5, based upon the var1CSA DBL3-γ minimal binding domain. The code for the consensus residues is h (hydrophobic), a (aromatic), − (negative charge), and 1 (aliphatic). A 67 amino acid minimal CSA binding region has been defined from the FCR3 var1CSA DBL3-γ domain (Gamain et al. Mol Micro 2004. 53:445-455). The 67 residue minimal binding region maps to the C-terminal region of the FCR3 var1CSA DBL3-γ domain. The 67 residue minimal binding region has homology to other DBL-γ domains that bind CSA (Gamain et al. 2004 Mol Microbiol 53:445-455) and to the CSA-binding DBL domains in the var2CSA (PFL0030c-like genes). A canonical sequence for CSA-binding domains based upon the minimal binding region from FCR3 var1CSA DBL3-γ and other domains that bound CSA (FIG. 1) would be defined as follows (with the single letter code with X meaning any amino acid): (hydrophobic)XEWX(E/D)X(F/Y)(C1)X₂RX₆(aliphatic)X₃C2(variable length with one or three cysteines)C4X₃C5X₂YX₂(aromatic)(aliphatic)(variable length)(aromatic)X_{6/7}(F/Y)X₈ (SEQ ID NO: 1).

FIG. 4. Targeted gene disruption of Plasmodium falciparuin var2csa. (A) Schematic representation of the disruption of var2csa by double-crossover integration. The pHTK-var2csa plasmid contains the thymidine kinase gene, hDHFR, and the sequences corresponding to DBL3-X and DBL5-ε of var2csa. The DBL4-ε region has been deleted and replaced by the hDHFR gene. The different Duffy binding-like (DBL) domains and the transmembrane (TM) domain, and carboxy-terminal cytoplasmic domain (ATS) of var2csa are shown. Homologous target sequences are shown in dark grey. Sizes of DNA fragments are shown in kilobases (kb). Restriction enzyme sites and the expected restriction fragments are indicated. Hybridization probes are indicated as black bars. (B) Knockout of var2csa by a double-crossover event. Southern blot analysis of genomic DNA from representative mutant clones 1F1 and 2A5 and the parental FCR3 strain using BamHI, EcoRV and PvuII restriction enzymes. Hybridization was carried out with DBL3-X- and DBL5-ε-specific probes. The positions of the probes are shown in (A). (C) Insertion of pHTK-var2csa in chromosome 12. Southern blot analysis of chromosomal DNA derived from FCR3 wt (wild type) and the representative disrupted mutant clones 1F1 and 2A5. Chromosomes were separated by pulsed-field gel electrophoresis, then transferred onto a nylon membrane and hybridized with probes specific for DBL5-ε, hDHFR and clathrin heavy chain. The position of chromosome 12 is indicated.

FIG. 5. FCR3Δvar2csa clones cytoadhere to CD36 and express a var gene that is different from var2csa. (A) Cytoadhesion of the var2csa disruption mutants to receptors coated to plastic Petri dishes. Erythrocytes infected with the Plasmodium falciparum FCR3-CSA, FCR3-CD36 and the FCR3Δvar2csa clones 1F1 and 2A5 were analyzed for cytoadhesion to CSA and CD36. Data are the mean±s.e.m. of IE per square millimeter (IE/mm²) adhering to CSA-coated (left panel) and CD36-coated (right panel) plastic Petri dishes, as determined in three independent assays. (B) Northern analyses of total RNA isolated from ring-stage (R) and trophozoite-stage parasites (T) FCR3-CSA, FCR3-CD36 and the representative FCR3Δvar2csa clones 1F1 and 2A5. The membrane was hybridized with a probe specific for var2csa DBL3-X and semiconserved varT11.1 exon II.

FIG. 6. FCR3Δvar2csa mutants show no chondroitin sulphate A (CSA)-specific cytoadhesion after selection on CSA-expressing cell lines and recombinant human thrombomodulin. Mean×s.d. of IE adhering per square millimeter (IE/mm²) for four different fields is shown (A,B). (A) Selection of FCR3Δvar2csa mutants and parental FCR3 parasites on recombinant human thrombomodulin. Erythrocytes infected with FCR3-CSA, FCR3-CD36, 1F1 and 2A5 were selected four times on recombinant human thrombomodulin-CSA coated to Petri dishes. (B) Selection of FCR3Δvar2csa mutants and parental FCR3 parasites on Sc1707. Trophozoite-stage Plasmodium falciparum clones FCR3-CSA, FCR3-CD36, 1F1 and 2A5 were subjected to repeated rounds of selection over Sc1707 cells, followed by evaluation of adhesion to Sc1707. (C,D) Adhesion profiles of P. falciparum IE after selection on Sc1707 cells. Trophozoite-stage P. falciparum parasites FCR3-CSA, FCR3-CD36, 1F1 and 2A5 were subjected to repeated rounds of selection over Sc1707 cells, followed by evaluation of adhesion to CSA-coated (C) and CD36-coated (D) plastic Petri dishes. Adhesion after selection is shown for FCR3-CSA¹⁷⁰⁷, FCR3-CD36¹⁷⁰⁷, 1F1¹⁷⁰⁷ and 2A5¹⁷⁰⁷. Data are the mean±s.e.m. of IE per square millimeter adhering to CSA- and CD36-coated plastic Petri dishes, as determined in three independent assays.

### DETAILED DESCRIPTION OF THE PREFERRED EMBODIMENT

Malaria in pregnancy is a serious complication associated with parasitized erythrocyte (PE) sequestration in the placenta. Recent work suggests that var genes could have an important role in PE binding to chondroitin sulfate A (CSA), a primary placental adherence receptor. Here we confirm that var2CSA is transcriptionally upregulated in CSA-binding parasites and define for the first time CSA-binding domains in var2CSA. The identification of multiple binding domains in var2CSA is envisioned as forming the basis of a vaccine against malaria especially in pregnancy.

### DEFINITIONS

Unless defined otherwise, technical and scientific terms used herein have the same meaning as commonly understood by one of ordinary skill in the art to which this invention belongs. See, e.g., Singleton P. and Sainsbury D., Dictionary of Microbiology and Molecular Biology 3^{rd} ed., J. Wiley & Sons, Chichester, N.Y., 2001.

CSA-Binding Domains Based Upon a Minimal Binding Domain and Other Domains that Bind CSA

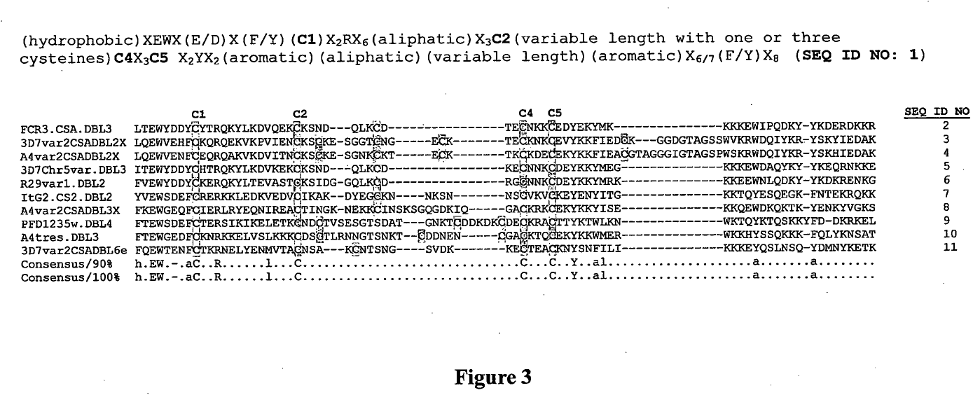

In one aspect the invention provides a polypeptide comprising the canonical sequence substantially as shown in FIG. 3, (SEQ ID NO: 1), or functional equivalents thereof, substantially (within 1-10 amino acids) in isolation from sequences naturally occurring adjacent thereto in the PfEMP1 protein.

In one aspect the invention provides a polypeptide comprising the FCR3.CSA.DBL3 sequence substantially as shown in FIG. 3, (SEQ ID NO: 2), or at least 95% identical thereto, or functional equivalents thereof, substantially in isolation from sequences naturally occurring adjacent thereto in the PfEMP1 protein.

In one aspect the invention provides a polypeptide comprising the 3D7var2CSADBL2X sequence substantially as shown in FIG. 3, (SEQ ID NO: 3), or at least 95% identical thereto, or functional equivalents thereof, substantially in isolation from sequences naturally occurring adjacent thereto in the PfEMP1 protein.

In one aspect the invention provides a polypeptide comprising the A4var2CSADBL2X sequence substantially as shown in FIG. 3, (SEQ ID NO: 4), or at least 95% identical thereto, or functional equivalents thereof, substantially in isolation from sequences naturally occurring adjacent thereto in the PfEMP1 protein.

In one aspect the invention provides a polypeptide comprising the 3D7Chr5var.DBL3 sequence substantially as shown in FIG. 3, (SEQ ID NO: 5), or at least 95% identical thereto, or functional equivalents thereof, substantially in isolation from sequences naturally occurring adjacent thereto in the PfEMP1 protein.

In one aspect the invention provides a polypeptide comprising the R29var1.DBL2 sequence substantially as shown in FIG. 3, (SEQ ID NO: 6), or at least 95% identical thereto, or functional equivalents thereof, substantially in isolation from sequences naturally occurring adjacent thereto in the PfEMP1 protein.

In one aspect the invention provides a polypeptide comprising the ItG2.Cs2.DBL2 sequence substantially as shown in FIG. 3, (SEQ ID NO: 7), or at least 95% identical thereto, or functional equivalents thereof, substantially in isolation from sequences naturally occurring adjacent thereto in the PfEMP1 protein.

In one aspect the invention provides a polypeptide comprising the A4var2CSADBL3X sequence substantially as shown in FIG. 3, (SEQ ID NO: 8), or at least 95% identical thereto, or functional equivalents thereof, substantially in isolation from sequences naturally occurring adjacent thereto in the PfEMP1 protein.

In one aspect the invention provides a polypeptide comprising the PFD1235w.DBL4 sequence substantially as shown in FIG. 3, (SEQ ID NO: 9), or at least 95% identical thereto, or functional equivalents thereof, substantially in isolation from sequences naturally occurring adjacent thereto in the PfEMP1 protein.

In one aspect the invention provides a polypeptide comprising the A4tres.DBL3 sequence substantially as shown in FIG. 3, (SEQ ID NO: 10), or at least 95% identical thereto, or functional equivalents thereof, substantially in isolation from sequences naturally occurring adjacent thereto in the PfEMP1 protein.

In one aspect the invention provides a polypeptide comprising the 3D7var2CSADBL6s sequence substantially as shown in FIG. 3, (SEQ ID NO: 11), or at least 95% identical thereto, or functional equivalents thereof, substantially in isolation from sequences naturally occurring adjacent thereto in the PFEMP1 protein.

FIG. 3 shows the amino acid sequences of the CSA-binding domains of the polypeptide known as PfEMP1 corresponding to the var1CSA DBL3-γ minimal binding domain. It is clear to those skilled in the art that minor alterations can be made to the sequence of the CSA-binding domains without significantly altering the biological properties thereof, so as to result in a functional equivalent.

For example, as well as allelic variants, functional equivalents might include those in which there are one or more conserved amino acid substitutions (i.e., the substitution of an amino acid for one with similar properties). Other substitutions that could be made are those that change an amino acid from one DBL sequence to that from another DBL sequence that substantially preserve the DBL structure. Alternatively, or in addition, minor additions, deletions or truncations of the CSA-binding domains could be made. Other obvious functional equivalents are those CSA-binding domains present in the PfEMP1 protein of other species of Plasmodium (such as the four species known to infect humans, the simian pathogen P. reichenowi, or the mouse pathogen P. yoelii).

### Expression of CSA-Binding Domains

The sequence of many of the var genes encoding PfEMP have been determined. Those with ordinary skill in the art could readily determine the sequence of other var genes. The inventors were able to identify a canonical sequence for a CSA-binding domain in the PfEMP1 protein based upon the minimal binding region from FCR3 var1CSA DBL3-γ and other domains that bound CSA (FIG. 3).

The nucleotide sequences encoding CSA-binding domains were inserted into vectors. The resulting plasmid constructs were able to direct the expression of the CSA-binding domains. Thus in another aspect the invention provides a vector comprising the nucleotide sequence encoding CSA-binding domains substantially in isolation from sequences naturally occurring adjacent thereto in the PfEMP1 proteins.

Generally the vector defined above is capable of expressing the CSA-binding domains.

The CSA-binding domains may be expressed in such a way that they retain the conformation that they adopt in the PfEMP1 proteins.

In another aspect the invention provides a method of producing the CSA-binding domains substantially in isolation from other PfEMP1 sequences, in the conformation they adopt in PfEMP1, comprising inserting the vector defined above capable of expressing the CSA-binding domains in a suitable host cell, growing the host cell and isolating the CSA-binding domains so produced.

In a further aspect the invention provides a nucleotide sequence comprising the sequence encoding the CSA-binding domains or functional equivalents thereof substantially in isolation from other PfEMP1-encoding nucleotide sequences. Such functional equivalents include those sequences that whilst possessing a different nucleotide sequence, by virtue of the degeneracy of the genetic code, encode the same amino acid sequence (or an amino acid sequence containing conserved substitutions or minor deletions, additions or truncations) and those nucleotide sequences that hybridize to the complement of the nucleotide sequence of the invention.

Advantageously the CSA-binding domains are expressed as naked DNA or as recombinant proteins with no additional sequence or as fusion proteins.

Conveniently the fusion protein should be one that allows for ease of purification such as fusion with glutathione S-transferase. Other such readily-purified fusion proteins are known to those skilled in the art.

Alternatively the fusion protein should be one that self-assembles into particles, such as described in PCT/US01/25625, in which the CSA-binding domain is expressed on the surface of the particles.

Alternatively, the antigen is attached to self-assembled particles, such as described in PCT/IB99/01925.

In another embodiment, the CSA-binding domains are expressed as cyclic proteins. Use of a cyclic protein is thought to be superior to a linear protein because the cyclic protein is able to assume fewer conformations than the linear protein and is therefore structurally more constrained. Typically, the cyclic protein comprises a cyclized portion, which cyclized portion preferably comprises an amino acid sequence, the terminal amino acids of which are linked together by a covalent bond. The covalent bond is conveniently a disulphide bridge, such as found between cysteine residues. The cyclized portion typically comprises the CSA-binding domain and the CSA-binding domain can conveniently form part of the amino acid sequence that is flanked by the amino acids that are linked by the covalent bond to form the cyclized portion.

In a further aspect the invention provides a host cell transformed with the vector defined above. The transformed host cell may be of bacterial, plant, fungal or animal origin.

The invention may be used for vaccine, diagnostic, functional (e.g., molecular decoy), or serologic purposes.

### Vaccines and Immunogenic Compositions

Expression of the CSA-binding domains in their native conformation (as judged by their reaction with antibodies that are known to inhibit CSA-binding or antibodies that react with CSA-binding parasite lines or functional criteria such as the ability to bind CSA or to compete with infected erythrocyte for binding to CSA) may allow for the use of recombinant DNA-derived material as a vaccine to induce a protective immune response against malaria.

Thus in another aspect the invention provides a vaccine comprising the sequences as described herein or functional equivalents thereof. Advantageously the vaccine comprises multiple CSA-binding domains or functional equivalents thereof. Conveniently the vaccine comprises the polypeptide in its native conformation and is generally administered with an appropriate adjuvant (e.g., alum or others that mediate monocyte opsinization in the placenta). Typically the CSA-binding domains will be carried in a physiologically acceptable carrier and/or be fused to another immunogen.

In another aspect the invention provides a method of treating a human body by administering a vaccine comprising the sequences as described herein or functional equivalents thereof.

### Var2CSA Binding Domains

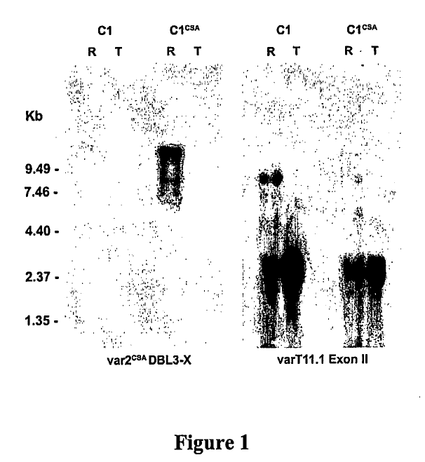

To determine the role of var2CSA in CSA adhesion, Northern blot analysis was performed on a parasite line in which var1CSA had been genetically disrupted (C1 mutant) (Andrews K T et al. 2003 Mol Miciobiol 49:655-669). C1 parasites bind CD36 (Andrews K T et al. 2003 Mol Microbiol 49:655-669) and predominantly express an 8-9 kb var product (FIG. 1). Upon CSA selection, the C1-CSA parasite line switches to the larger, var2CSA transcript (FIG. 1), confirming previous data that var2CSA is transcriptionally upregulated in different parasite strains selected to bind CSA (Salanti A et al. 2003 Mol Microbiol 49:179-191).

3D7 var2CSA contains three DBL-X type and three DBL-E type domains (Salanti A et al. 2003 Mol Microbiol 49:179-191). To determine the CSA binding domain(s) from var2CSA, recombinant proteins corresponding to the different 3D7 var2CSA individual domains were expressed on the surface of CHO-745 cells and tested for binding. 3D7 var2CSA DBL2-X and DBL6-s bound Biot-CSA (Table 1). The binding was specific to CSA as the adhesion of Biot-CSA was inhibited by soluble CSA but not by soluble CSC (Table 1). We also tested the DBL2-X domain of A4 var2CSA. Like 3D7, it bound to Biot-CSA (Table 1). None of the domains tested bound Biot-CSC.

TABLE 1
Binding Characteristics of domains from var2CSA to Biot-CSA

| | Binding of Biot-CSA to CHO-745 cells* | | | | | |
|---|---|---|---|---|---|---|
| | No inhibition | | Inhibition with CSA | | Inhibition with CSC | |
| Construct | Positive | Beads/100 | Positive | Beads/100 | Positive | Beads/100 |
| expressed | cells^{†} (%) | cells^{‡} | cells^{†} (%) | cells^{‡} | cells^{†} (%) | cells^{‡} |
| 3D7-DBL1-X | 1 ± 2 | ND | 2 ± 1 | ND | 1 ± 1 | ND |
| 3D7-DBL2-X | 88 ± 7 | 1217 ± 73 | 4 ± 2 | ND | 82 ± 4 | 1179 ± 89 |
| 3D7-DBL3-X | 2 ± 1 | ND | 1 ± 0 | ND | 1 ± 0 | ND |
| 3D7-DBL3b-X | 0 ± 1 | ND | 1 ± 1 | ND | 1 ± 1 | ND |
| 3D7-DBL4-ε | 2 ± 2 | ND | 1 ± 0 | ND | 1 ± 2 | ND |
| 3D7-DBL5-ε | 1 ± 1 | ND | 0 ± 0 | ND | 1 ± 0 | ND |
| 3D7-DBL6-ε | 75 ± 6 | 1156 ± 52 | 19 ± 8 | ND | 70 ± 9 | 1027 ± 21 |
| A4-DBL1-X | 1 ± 2 | ND | 1 ± 1 | ND | 0 ± 0 | ND |
| A4-DBL2-X | 93 ± 5 | 1398 ± 79 | 3 ± 1 | ND | 91 ± 6 | 1239 ± 56 |
| A4-DBL3-X | 87 ± 4 | 1306 ± 67 | 2 ± 2 | ND | 85 ± 11 | 1269 ± 78 |
| A4-DBL3b-X | 84 ± 7 | 1253 ± 92 | 4 ± 3 | ND | 81 ± 6 | 1125 ± 48 |

*Cells were incubated with Biot-CSA without (control) or after preincubation with 200 μg/ml CSA or CSC.
^{†}One hundred cells expressing the recombinant protein were evaluated for the presence of CSA-coated Dynal beads on their surface. Cells with four or more beads attached were considered positive for binding. Results are expressed as the mean and SD of three independent experiments.
^{‡}The number of beads was counted on 100 cells determined positive for binding to CSA-coated Dynal beads. Results are expressed as the mean and SD of three independent experiments.

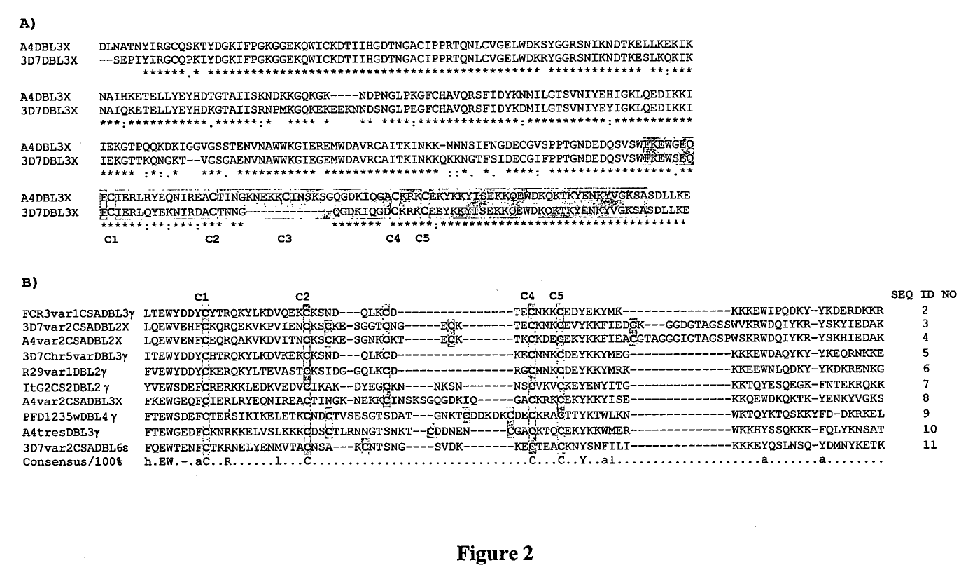

Interestingly, the 3D7 var2CSA DBL3-X domain was negative for CSA binding, although this domain had the best sequence similarities with the minimal CSA binding region from the FCR3 var1CSA DBL3 γ domain (Gamain B. et al. 2004 Mol Microbiol 53:445-455). This same domain still did not bind Biot-CSA when expressed as a slightly longer domain (3D7 DBL3b-X, Table 1). Sequence comparison of var2CSA from A4, MC, 3D7 and Dd2 strains (Kraemer S. M. et al. 2003 Mol Microbiol 50:1527-1538) showed that DBL3-X domains were highly similar (80% homology) but contained a 12 residue deletion in the 3D7 DBL3-X (FIG. 2A). This modification eliminated, among other things, a cysteine residue (FIGS. 2A and B) present in the var1CSA DBL3 γ minimal binding domain and might explain the inability of the 3D7 DBL3-X to bind CSA. To examine this hypothesis, the DBL3-X and DBL3b-X domains from the A4 var2CSA were expressed on CHO cells and tested for binding (Table 1). The A4 DBL3-X and DBL3b-X domains bound specifically to Biot-CSA (Table 1), but did not bind Biot-CSC. Considered with the previous mapping of the minimal binding domain in var1CSA (Gamain B. et al. 2004 Mol Microbiol 53:445-455), this var2CSA binding comparison additionally highlights the potential importance of the C-terminal region in CSA binding DBL domains. Further studies such as those described herein and others well known to those in the art are expected to confirm the minimal domain (s) of DBL2-X, DBL3-X and DBL6-ε responsible for the CSA binding phenotype.

Since the initial description that maternal antibodies recognize placental isolates from geographically dispersed regions and block infected erythrocyte binding to CSA (Fried M. et al. 1998 Nature 395:851-852) there has been intense interest to define the parasite adhesion ligands, as these may form the basis of a pregnancy malaria vaccine. A leading candidate has been the parasite variant surface antigens responsible for cytoadhesive activities, but questions have arisen whether these are too divergent between parasite strains to explain the cross-reactive antibody response. Unlike the majority of the var gene family, var2CSA sequences are unusually conserved between parasite strains (Kraemer S. M. et al. 2003 Mol Microbiol 50:1527-1538; Salanti A. et al. 2003 Mol Microbiol 49:179-191). In this study, we demonstrate that two slightly distinct var2CSA sequences, which are transcriptionally upregulated in different CSA-adherent parasite strains, contain multiple CSA-binding domains. This is the first evidence that var2csa sequences bind CSA and raises the possibility that multivalency is important for infected erythrocyte sequestration in the placenta. Including this study and others (Gamain B. et al. 2004 Mol Microbiol 53:445-455), several different DBL domains including DBL-γ, ε, and X types have now been shown to specifically bind CSA (FIG. 2B). Binding sequences are highly diverse, although a consensus sequence based upon the minimal binding region from FCR3 var1CSA DBL3-γ and other CSA-binding domains (FIG. 2B) would be defined as follows (with the single letter code with X meaning any amino acid): (hydrophobic)XEWX(E/D)X(F/Y)(C1)X₂RX₆(aliphatic)X3(C2)(variable length with one or three cysteines)(C4)X₃(C5)X₂YX₂(aromatic)(aliphatic)(variable length)(aromatic)X_{6/7}(F/Y)X₈. These consensus residues can also be detected in DBLγ domains that do not bind CSA (Gamain B. et al. 2004 Mol Microbiol 53:445-455), suggesting that they are necessary but not sufficient for binding.

In conclusion, the var2CSA is a strain-transcendent member of the parasite variant antigen family, which is transcriptionally upregulated in infected erythrocytes selected to bind CSA. Identification of CSA binding domains in var2CSA strengthens the evidence for their involvement in malaria during pregnancy and is envisioned as forming the basis of broad-spectrum vaccine(s) against malaria especially in pregnancy.

TABLE 2
Var2CSA-Binding DBL Domains

| | | SEQ |
|---|---|---|
| DBL Domain | Domain Boundaries (amino acids) | ID NO |
| 3D7-DBL2-X | 542-853 (of Accession Number NP_701371) | 12 |
| A4-DBL2-X | 543-858 (of Accession Number AAQ73926) | 13 |
| A4-DBL3-X | 1220-1541 (of Accession Number AAQ73926) | 14 |
| 3D7-DBL6-ε | 2318-2589 (of Accession Number NP_701371) | 15 |

### Functional Fragments of CSA-Binding DBL Domains

Embodiments also include polypeptides that comprise CSA-binding sequences or fragments thereof. These polypeptide embodiments can be for example, at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 225, 250, 275, and 300 amino acids in length so long as the polypeptide can bind CSA (as judged by their reaction with antibodies that are known to inhibit CSA-binding or antibodies that react with CSA-binding parasite lines or functional criteria such as the ability to bind CSA or to compete with infected erythrocyte for binding to CSA). As with other polypeptide embodiments described herein, the polypeptides comprise 3D7-DBL2-X, (SEQ ID NO: 12), A4-DBL2-X (SEQ ID NO: 13), A4-DBL3-X (SEQ ID NO: 14), and 3D7-DBL6-ε (SEQ ID NO: 15) sequence or fragments thereof.

Embodiments further include nucleic acids encoding polypeptides that comprise CSA-binding sequences or fragments thereof. These nucleic acid embodiments can be for example, at least 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 125, 150, 175, 200, 250, 300, 350, 400, 450, 500, 550, 600, 650, 700, 750, 800, 850, and 900 nucleotides in length so long as the polypeptide it encodes can bind CSA (as judged by their reaction with antibodies that are known to inhibit CSA-binding or antibodies that react with CSA-binding parasite lines or functional criteria such as the ability to bind CSA or to compete with infected erytbrocyte for binding to CSA). As with other nucleic acid embodiments described herein, the nucleic acids encoding polypeptides that comprise 3D7-DBL2-X, (SEQ ID NO: 12), A4-DBL2-X (SEQ ID NO: 13), A4-DBL3-X (SEQ ID NO: 14), and 3D7-DBL6-6 (SEQ ID NO: 15) sequence or complements thereto or fragments thereof can be incorporated into vectors, plasmids, expression constructs and organisms, including humans.

### Polypeptides and Fragments

The invention further provides a polypeptide having the amino acid sequence as described herein or a portion of the above polypeptides.

It will be recognized in the art that some amino acid sequences of the CSA binding domain can be varied without significant effect of the structure or function of the domain. If such differences in sequence are contemplated, it should be remembered that there will be critical areas on the domain that determine activity.

Thus, the invention further includes variations of the CSA binding domain that show substantial CSA binding activity or that include regions of the domain such as the portions discussed below. Such mutants include deletions, insertions, inversions, repeats, and type substitutions. As indicated, guidance concerning which amino acid changes are likely to be phenotypically silent can be found in Bowie, J. U. et al. 1990 Science 247:1306-1310.

Thus, the fragment, derivative or analog of the CSA binding domain, may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which additional amino acids are fused to the CSA binding domain such as a leader or secretory sequence or a sequence that is employed for purification of the mature polypeptide or a proprotein sequence. Such fragments, derivatives and analogs are deemed to be within the scope of those skilled in the art from the teachings herein.

As indicated, changes are preferably of a minor nature, such as conservative amino acid substitutions that do not significantly affect the folding or activity of the CSA binding domain (see Table A).

TABLE A
Conservative Amino Acid Substitutions.

| | |
|---|---|
| Aromatic | Phenylalanine |
| | Tryptophan |
| | Tyrosine |
| Hydrophobic | Leucine |
| | Isoleucine |
| | Valine |
| Polar | Glutamine |
| | Asparagine |
| Basic | Arginine |
| | Lysine |
| | Histidine |
| Acidic | Aspartic Acid |
| | Glutamic Acid |
| Small | Alanine |
| | Serine |
| | Threonine |
| | Methionine |
| | Glycine |

Of course, the number of amino acid substitutions a skilled artisan would make depends on many factors, including those described above. Generally speaking, the number of amino acid substitutions for any given CSA binding domain will not be more than 50, 40, 30, 20, 10, 5 or 3.

Amino acids in the CSA binding domain of the present invention that are essential for function can be identified by methods known in the art, such as site-directed mutagenesis or alanine-scanning mutagenesis (Cunningham and Wells, 1989 Science 244:1081-1085). The latter procedure introduces single alanine mutations at every residue in the molecule. The resulting mutant molecules are then tested for biological activity such as CSA binding activity.

The polypeptides of the present invention are preferably provided in an isolated form. By “isolated polypeptide” is intended a polypeptide removed from its native environment. Thus, a polypeptide produced and/or contained within a recombinant host cell is considered isolated for purposes of the present invention.

Also intended as an “isolated polypeptide” are polypeptides that have been purified, partially or substantially, from a recombinant host cell or a native source. For example, a recombinantly produced version of the CSA binding domain can be substantially purified by the one-step method described in Smith and Johnson, 1988 Gene 67:31-40.

The polypeptides of the present invention include a polypeptide having the amino acid sequence of a CSA binding domain as described herein; as well as polypeptides that are at least 95% identical, and more preferably at least 96%, 97%, 98% or 99% identical to those described above and also include portions of such polypeptides with at least 30 amino acids and more preferably at least 50 amino acids.

By a polypeptide having an amino acid sequence at least, for example, 95% “identical” to a reference amino acid sequence of a CSA binding domain is intended that the amino acid sequence of the polypeptide is identical to the reference sequence except that the polypeptide sequence may include up to five amino acid alterations per each 100 amino acids of the reference amino acid of the CSA binding domain. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 95%, 96%, 97%, 98% or 99% identical to a reference amino acid sequence can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

### Nucleic Acid Molecules

As indicated, nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) encoding a CSA binding domain; and DNA molecules that comprise a sequence substantially different from those described above but that, due to the degeneracy of the genetic code, still encode a CSA binding domain. Of course, the genetic code is well known in the art. Thus, it would be routine for one skilled in the art to generate such degenerate variants.

The present invention is further directed to fragments of the isolated nucleic acid molecules described herein. By a fragment of an isolated nucleic acid molecule having the nucleotide sequence of an ORF encoding a CSA binding domain is intended fragments at least about 15 nt, and more preferably at least about 20 nt, still more preferably at least about 30 nt, and even more preferably, at least about 40 nt in length. Of course, larger fragments 50, 100, or 150 nt in length are also useful according to the present invention as are fragments corresponding to most, if not all, of the nucleotide sequence of the ORP encoding a CSA binding domain. By a fragment at least 20 nt in length, for example, is intended fragments that include 20 or more contiguous bases from the nucleotide sequence of the ORF encoding a CSA binding domain.

Preferred nucleic acid fragments of the present invention include nucleic acid molecules encoding a minimal binding domain.

In another aspect, the invention provides an isolated nucleic acid molecule comprising a polynucleotide that hybridizes under stringent hybridization conditions to a portion of the polynucleotide in a nucleic acid molecule of the invention described above. By “stringent hybridization conditions” is intended overnight incubation at 42° C. in a solution comprising: 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 65° C.

By a polynucleotide that hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 nt of the reference polynucleotide.

By a portion of a polynucleotide of “at least 20 nt in length,” for example, is intended 20 or more contiguous nucleotides from the nucleotide sequence of the reference polynucleotide. Of course, a polynucleotide that hybridizes only to a poly A sequence, or to a complementary stretch of T (or U) resides, would not be included in a polynucleotide of the invention used to hybridize to a portion of a nucleic acid of the invention, since such a polynucleotide would hybridize to any nucleic acid molecule containing a poly (A) stretch or the complement thereof (e.g., practically any double-stranded cDNA clone).

As indicated, nucleic acid molecules of the present invention that encode a CSA binding domain may include, but are not limited to those encoding the amino acid sequence of the full-length domain, by itself, the coding sequence for the full-length domain and additional sequences, such as those encoding a leader or secretory sequence, such as a pre-, or pro- or prepro-protein sequence, the coding sequence of the full-length domain, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example—ribosome binding and stability of mRNA; and additional coding sequence that codes for additional amino acids, such as those that provide additional functionalities.

The present invention further relates to variants of the nucleic acid molecules of the present invention, which encode portions, analogs or derivatives of the CSA binding domain. Variants may occur naturally, such as a natural allelic variant. By an “allelic variant” is intended one of several alternate forms of a gene occupying a given locus on a chromosome of an organism (Genes II, Lewin, B., ed., John Wiley & Sons, New York 1985). Non-naturally occurring variants may be produced using art-known mutagenesis techniques.

Such variants include those produced by nucleotide substitutions, deletions or additions, which may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the CSA binding domain or portions thereof. Also especially preferred in this regard are conservative substitutions.

Further embodiments of the invention include isolated nucleic acid molecules comprising a polynucleotide having a nucleotide sequence at least 95% identical, and more preferably at least 96%, 97%, 98% or 99% identical to a nucleotide sequence encoding the CSA binding domain or fragment thereof or a nucleotide sequence complementary thereto.

By a polynucleotide having a nucleotide sequence at least, for example, 95% “identical” to a reference nucleotide sequence encoding a CSA binding domain is intended that the nucleotide sequence of the polynucleotide is identical to the reference sequence except that the polynucleotide sequence may include up to five point mutations per each 100 nucleotides of the reference nucleotide sequence encoding the CSA binding domain. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 95%, 96%, 97%, 98% or 99% identical to the reference nucleotide sequence can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, 1981 Advances in Applied Mathematics 2: 482-489, to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The present application is directed to nucleic acid molecules at least 95%, 96%, 97%, 98% or 99% identical to the nucleic acid sequences described herein that encode a CSA binding domain. By “CSA binding domain” is intended domains that exhibit CSA binding activity in a particular biological assay. For example, CSA binding activity can be measured using the binding assay described in Buffet P. A. et al. 1999 PNAS USA 96:12743-12748; and Gamain B. et al. 2004 Mol Microbiol 53:445-455.

Of course, due to the degeneracy of the genetic code, one of ordinary skill in the art will immediately recognize that a large number of the nucleic acid molecules having a sequence at least 95%, 96%, 97%, 98%, or 99% identical to a nucleic acid sequence described herein will encode a polypeptide “having a CSA binding activity”. In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having a CSA binding activity. This is because the skilled artisan is fully aware of amino acid substitutions that are either less likely or not likely to significantly effect protein function (e.g., replacing one aliphatic amino acid with a second aliphatic amino acid).

For example, guidance concerning how to make phenotypically silent amino acid substitutions is provided in Bowie, J. U. et al. 1990 Science 247:1306-1310, wherein the authors indicate that proteins are surprisingly tolerant of amino acid substitutions.

### Canonical Sequence of CSA-Binding Domains

A 67 amino acid minimal CSA binding region has been defined from the FCR3 var1CSA DBL3-γ domain (Gamain, B. et al. 2004 Mol Microbiol 53:445-455). The 67 residue minimal binding region maps to the C-terminal region of the FCR3 var1CSA DBL3-γ domain.

The 67 residue minimal binding region has homology to other DBL-γ domains that bind CSA (Gamain, B. et al. 2004 Mol Microbiol 53:445-455) and to the CSA-binding DBL domains in the var2CSA (PFL0030c-like genes) (FIG. 2). A canonical sequence for CSA-binding domains based upon the minimal binding region from FCR3 var1CSA DBL3-γ and other domains that bound CSA (FIG. 3) would be defined as follows (with the single letter code with X meaning any amino acid): (hydrophobic)XEWX(E/D)X(F/Y)(C1)X₂RX₆(aliphatic)X3(C2)(variable length with one or three cysteines)(C4)X₃(C5) X₂YX₂(aromatic)(aliphatic)(variable lengh)(aromatic)X_{6/7}(F/Y)X₈

### Example 1

### Var2CSA Northern Blot Analysis

Total RNA was prepared from ring and trophozoite stages cultures approximately 10 h and 24 h post-invasion, respectively. RNA preparation, electrophoresis, membrane transfer (Hybond N+, Amersham) and hybridization with radiolabelled A4 PFL0030c DBL3 probe (3660-4623 bp) or a probe based upon the var semi-conserved exon 2 (var T11.1 gene, GenBank accession number U67959, 7930-9147 bp) generated using the megaprime Labelling Kit (Amersham) were carried out as previously described (Kyes S. et al. 2000 Mol Biochem Parasitol 105:311-315). Membranes were hybridized at high stringency conditions at 60° C. overnight and washed twice with 0.2×SSC, 0.1% SDS at 60° C. for 30 min.

### Surface Expression of Various Domains in CHO-745 Cells

Constructs were amplified from genomic DNA by PCR and cloned into the pSRα5 12CA5 vector (Affymax Research Institute), as described before (Gamain B. et al. 2004 Mol Microbiol 53:445-455). The following domains from 3D7 PFL0030c var2CSA (GenBank accession number NP_{—}701371) and from A4 var2CSA (GenBank accession number AAQ73926) were used (amino acid (aa) boundaries of each clone): 3D7 DBL1 (57-382); 3D7 DBL2 (542-853); 3D7 DBL3 (1213-1523); 3D7 DBL3b (1213-1571); 3D7 DBL4 (1576-1883); 3D7 DBL5 (2001-2272); 3D7 DBL6 (2318-2589); A4 DBL1 (52-383); A4 DBL2 (543-858); A4 DBL3 (1220-1541); A4 DBL3b (1220-1580). Chinese hamster ovary PgsA 745 (CHO-745) cells deficient in glycosaminoglycans (American Type Culture Collection) were transfected and selected by single cell cloning using a FACS sorter as described before (Gamain B et al. 2004 Mol Microbiol 53:445-455).

Binding Assays with CSA Linked to Biotin

Binding assays with Bovine trachea CSA (Sigma) or Shark cartilage CSC (Sigma or Seikagaku) linked to biotin (Biot CSA or Biot-CSC) were performed as previously described (Buffet P. A. et al. 1999 PNAS USA 96:12743-12748; Gamain B. et al. 2004 Mol Microbiol 53:445-455). In brief, 50 μg of Biot-CSA or Biot-CSC were incubated for 1 h with stably transfected CHO-745 clones grown on coverslips. Binding was visualized with Dynabeads (Dynal) coated with anti biotin mAb (Jackson Immunoresearch Labs). For inhibition assays, the cells were incubated for 1 h with 200 μg/ml of CSA or chondroitin sulfate C (CSC) (Sigma) before addition of Biot-CSA.

### Example 2

### A Single Member of the Plasmodium falciparum Var Multigene Family Determines Cytoadhesion to the Placental Receptor Chondroitin Sulphate A

In high-transmission regions, protective clinical immunity to Plasmodium falciparum develops during the early years of life, limiting serious complications of malaria in young children. Pregnant women are an exception and are especially susceptible to severe P. falciparum infections resulting from the massive adhesion of parasitized erythrocytes to chondroitin sulphate A (CSA) present on placental syncytiotrophoblasts. Epidemiological studies strongly support the feasibility of an intervention strategy to protect pregnant women from disease. However, different parasite molecules have been associated with adhesion to CSA. In this work, we show that disruption of the var2csa gene of P. falciparum results in the inability of parasites to recover the CSA-binding phenotype. This gene is a member of the var multigene family and was previously shown to be composed of domains that mediate binding to CSA. Our results show the central role of var2CSA in CSA adhesion and support var2CSA as the basis for a vaccine aimed at protecting pregnant women and their fetuses.

### Introduction

To assess the repertoire of CSA-binding ligands, we generated disruption mutants of the var2csa gene that was previously reported to possess several CSA-binding domains, and to be upregulated in placental parasites (see described above and in Salanti A. et al. 2003 Mol Microbiol 49:179-91). The FCR3Δvar2csa mutant parasites did not recover the CSA-binding phenotype, indicating that a single member of the P. falciparum var gene family determines cytoadhesion to CSA.

### Results

Targeted Disruption of the var2csa Gene in FCR3 Parasites

It has been reported that var2csa is transcriptionally upregulated and expressed at the surface of CSA-binding parasites (see results described above and in Salanti A. et al. 2003 Mol Microbiol 49:179-91; Salanti A. et al. 2004 J Exp Med 200:1197-203). To investigate the role of var2csa in P. falciparum IE adhesion to CSA, we established parasite lines with a disruption in the var2csa gene.

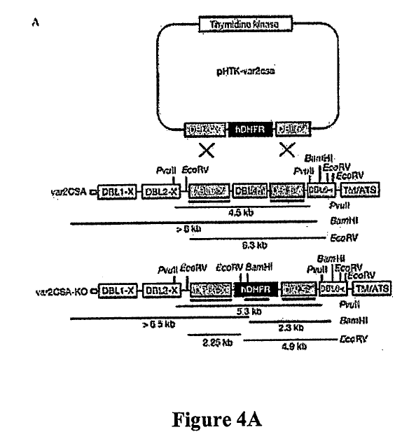

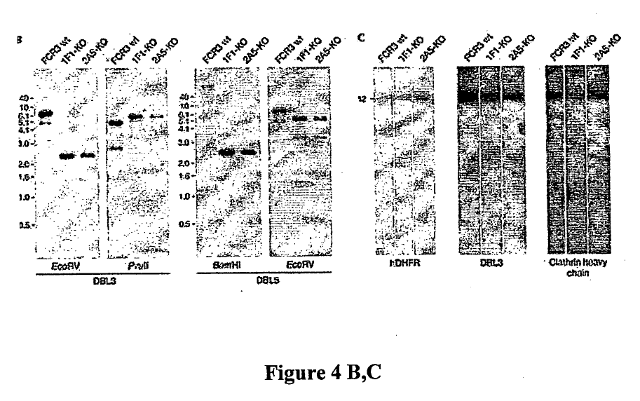

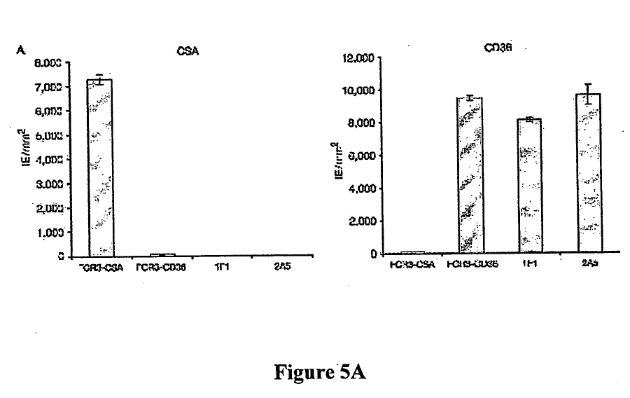

The pHTK-var2csa vector contains the hDHFR gene flanked by the var2csa DBL3-X and DBL5-ε sequences (FIG. 4A). Insertional disruptant mutants were generated by double-crossover homologous recombination of the pHTK-var2csa transfection construct, resulting in the replacement of the var2csa DBL4-c domain with the hDHFR expression cassette (FIG. 4A). FCR3 parasites were transfected with pHTK-var2csa and selected on WR99210 and ganciclovir to obtain FCR3Δvar2csa mutants. After selection of the FCR3Δvar2csa population for knob-positive parasites using gelatin flotation, the mutants were cloned by limiting dilution and genetically characterized.

Clones were screened by PCR analysis for the disruption of the var2csa gene as well as for the absence of contaminating wild-type var2csa and the presence of the HRP1 (KAHRP) gene. The presence of the HRP1 gene, taken together with the enrichment by gelatin flotation, argues for the presence of knobs on the surface of the FCR3Δvar2csa IE. To confirm that pHTK-var2csa had integrated into var2csa, Southern blots were performed using genomic DNA previously digested with BamHI, EcoRV or PvuII derived from parental FCR3 or recombinant parasites, and were hybridized with var2csa DBL3 or DBL5 radiolabelled probes (FIG. 4B). These hybridizations showed bands of the expected size, indicating that the integration occurred at the predicted site within the var2csa gene (FIG. 4A,B). Pulsed-field gel electrophoresis (PFGE) was performed to further support the integration of the selectable marker cassette within the var2csa locus on chromosome 12 (FIG. 4B). A clathrin heavy chain probe was used as a chromosome-12-specific marker. After the complete characterization of several mutant clones by PCR, and Southern blotting of both restriction enzyme digests and size-fractionated chromosomal DNA (FIG. 4B,C), two clones (1F1 and 2A5) were selected for further analysis.

FCR3Δvar2csa Clones Cytoadhere to CD36

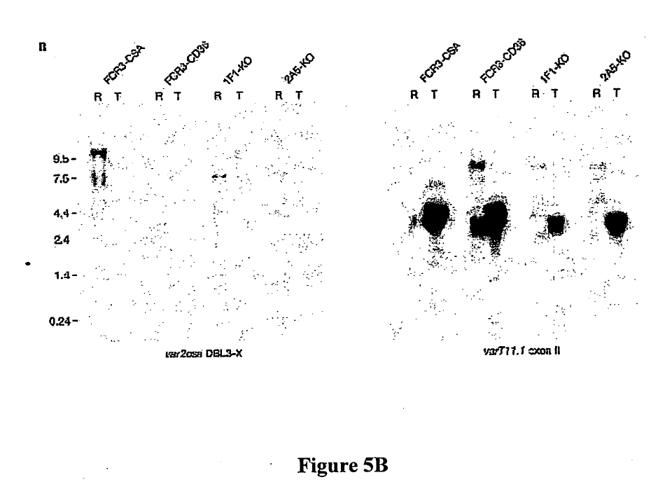

To test the ability of the FCR3Δvar2csa mutants to cytoadhere, adhesion of the FCR3Δvar2csa mutants to CSA and CD36 was examined (FIG. 5A). Equal numbers of erythrocytes infected with trophozoites of the FCR3Δvar2csa 1F1 and 2A5 mutant clones or control parasites were seeded on Petri dishes coated with different molecules. FCR3-CSA and FCR3-CD36 were used as controls. Whereas FCR3-CSA IE bound in high numbers to CSA but not to CD36, no adhesion to CSA was observed for 1F1, 2A5 and FCR3-CD36 IE. In contrast, 1F1, 2A5 and FCR3-CD36 IE adhered strongly to CD36. These results show that the FCR3Δvar2csa mutants are still able to mediate binding to another host receptor. No cytoadhesion to BSA and chondroitin sulphate C was observed.

Total RNA was isolated from ring- and trophozoite-stage parasites to investigate var gene expression in the FCR3Δvar2csa and the parental FCR3 parasites selected for a CSA- or CD36-binding phenotype. Whereas a full-length var2csa transcript (˜13 kb) was observed in the FCR3-CSA parasites, a nonfunctional truncated transcript (˜7 kb) was detected in the mutant clones 1F1 and 2A5 (FIG. 5B).

Using a semiconserved varT11.1 exon II probe, larger transcripts of around 9 kb were identified in ring-stage RNA of FCR3-CD36 and in the two mutant clones, showing that full-length var genes are transcribed in the CD36-binding FCR3Δvar2csa parasites. This result, taken together with the presence of a nonfunctional var2csa truncated transcript, shows that mutually exclusive transcription of var genes can be overcome under certain conditions. As blots were washed using high-stringency conditions, and because of the divergence in the var2csa exon II sequence, the var2csa transcript was not detected with the exon II probe in FCR3-CSA parasites. Sterile exon II transcripts were detected at the trophozoite stages for all the parasite clones (Su X. Z. et al. 1995 Cell 82:89-100). These results show that full-length var genes mediating CD36 binding are transcribed in the FCR3Δvar2csa clones 1F1 and 2A5 and that disruption of the var2csa locus does not interfere with IE cytoadhesion to receptors such as CD36.

No Adhesion of FCR3Δvar2csa Clones to CSA

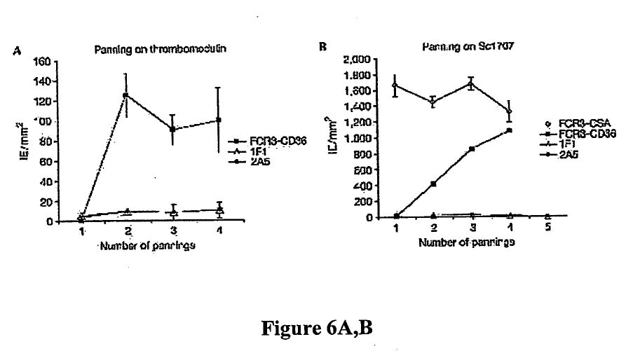

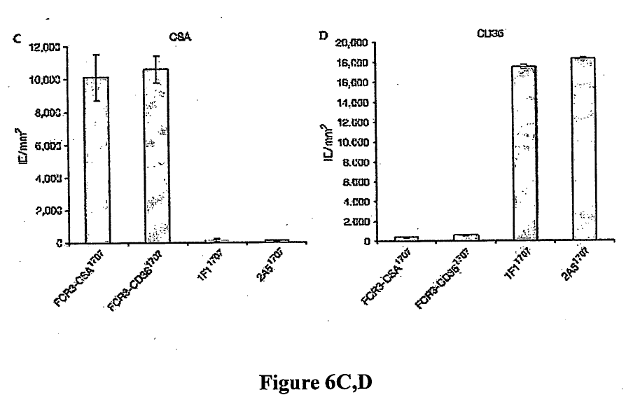

To determine the ability of the FCR3Δvar2csa mutants to recover cytoadherence to CSA, the parasites were re-selected on CSA using different systems. Switching of var genes occurs in in vitro-cultured parasites, and variants that are able to adhere to a large number of different host receptors have been isolated using receptor-specific panning assays (Roberts D. J. et al. 1992 Nature 357:689-92; Scherf A. et al. 1998 EMBO J 17:5418-26). FCR3Δvar2csa IE (clones 1F1 and 2A5) were first selected on recombinant human thrombomodulin-coated plastic dishes (Parzy D. et al. 2000 Microbes Infect 2:779-88). After four pannings, no specific enrichment was observed (FIG. 6A). However, FCR3-CD36 wild-type parasites could be selected for binding to CSA.

In addition, FCR3Δvar2csa E were selected on Saimiri brain microvasculature endothelial cell clone Sc1707, which was previously described to express exclusively the adhesion receptor CSA and to be a useful cell system for selecting CSA-binding parasites (Pouvelle B. et al. 1997 Mol Med 3:508-18). No adhesion of the FCR3Δvar2csa mutants to the Sc1707 cells was observed after five rounds of selection, whereas wild-type FCR3-CD36 population began to adhere to CSA after only one round of selection (FIG. 6B). Parasites selected on Sc1707 were renamed FCR3-CSA¹⁷⁰⁷, FCR3-CD36¹⁷⁰⁷, 1F1¹⁷⁰⁷ and 2A5¹⁷⁰⁷. Similar results were obtained by selecting the four parasite lines on CHO-K1 cells.

The adhesion phenotype of the Sc1707-selected parasites was examined on adhesion receptors coated to plastic Petri dishes. Whereas FCR3-CD36¹⁷⁰⁷ bound strongly to CSA, FCR3Δvar2csa clones 1F1¹⁷⁰⁷ and 2A5¹⁷⁰⁷ maintained the CD36-binding phenotype that had already been observed before the selection procedure (FIG. 6C,D). No CSA-specific adhesion was detected after five pannings of the FCR3Δvar2csa clones 1F1 and 2A5 on Sc1707. Taken together, our experiments suggest that the var2CSA protein is essential for cytoadhesion of late-stage FCR3-IE to CSA, as no other protein emerged to compensate var2CSA loss.

### Discussion

In conclusion, we show that a single member of the var repertoire is required for binding to CSA in FCR3 parasites. Given that FCR3Δvar2csa disruptant mutants do not recover this binding phenotype, even after several rounds of panning selection, we conclude that no other parasite gene can compensate for the loss of function under the experimental CSA selection conditions of our work. Thus, our demonstration of the central role of var2CSA in CSA adhesion is important for the future design of a vaccine against the complications of malaria during pregnancy.

### Methods

Parasites and cells. P. falciparum FCR3 clones were cultivated according to standard conditions (Trager W. & Jensen J. B. 1976 Science 193:673-5). Knob-positive parasites were routinely selected by gelatin flotation using Plasmion (Fresenius Kabi, France). Saimiri brain microvasculature endothelial cell clone Sc1707 was cultured, as described earlier (Pouvelle B. et al. 1997 Mol Med 3:508-18).

Plasmids and transfection. Fragments corresponding to the DBL3-X and DBL5-e domains of var2csa were PCR amplified from FCR3 genomic DNA using the primer combinations DBL3-F/DBL3-R and DBL5-F/DBL5-R. These PCR fragments were sequentially cloned into pHTK (Duraisingh M. T. et al. 2002 Int J Parasitol 32:81-9) using the SacII/SpeI sites, as well as the EcoRI/AvrII sites, to derive pHTK-var2csa.

Ring-stage FCR3 parasites were transfected with 100 μg plasmid DNA and selected with 2.5 nM WR99210 (Jacobus Pharmaceutical Co. Inc., Princeton, N.J., USA) and 4 μM ganciclovir (Sigma, Saint Quentin, Fallavier, France), as described previously (Duraisingh M. T. et al. 2002 Int J Parasitol 32:81-9).

Pulsed-field gel electrophoresis and Southern blot. Genomic DNA was digested and size fractionated on 0.8% agarose gels. PFGE and Southern blot were performed, as described previously (Hernandez-Rivas R. & Scherf A. 1997 Mem Inst Oswaldo Cruz 92:815-9). The chromosome-12-specific probe for clathrin heavy chain was obtained by PCR amplification using the primers CHC-F and CHC-R. The hDHFR probe was obtained by restriction of pHTK with BamHI and HindIII. Var2csa probes were used, as described in the Plasmids and transfection. Membranes were hybridized at high-stringency conditions at 60° C. overnight and washed twice with 0.2×SSC and 0.1% SDS at 60° C. for 30 min.

Northern blot analysis. Total RNA was prepared from synchronized parasite cultures approximately 10 and 30 h after invasion. RNA preparation, electrophoresis, membrane transfer and hybridization were carried out, as described previously (Kyes S. et al. 2000 Mol Biochem Parasitol 105:311-5). Membranes were hybridized at high-stringency conditions at 60° C. overnight and washed twice with 0.5×SSC and 0.1% SDS at 60° C. for 30 min. Radiolabelled probes for FCR3 var2csa DBL3-X or FCR3 var1csa DBL3-γ, or a probe based on the var semiconserved exon II (varT11.1 gene, 7,930-9,147 base pairs; GenBank accession number U67959) were generated, as described above.

P. falciparum adhesion assays and pannings. Trophozoite-stage IE were purified using gelatin flotation and selected for CSA binding on Sc1707 cells and on recombinant human thrombomodulin carrying CSA chains, as described previously (Pouvelle B. et al. 1997 Mol Med 3:508-18). For Sc1707, the number of IE bound per square millimeter was determined for four different fields and the mean±s.d. was calculated. Cytoadhesion assays on receptors immobilized on plastic Petri dishes were carried out, as described previously (Baruch D. I. et al. 1999 Blood 94:2121-7; Buffet P. A. et al. 1999 PNAS USA 96:12743-8). The average number of adherent IE (±standard error of the mean (s.e.m.)) for four different fields was determined in three independent experiments.

While the present invention has been described in some detail and form for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

1-46. (canceled)

47. An isolated polypeptide comprising a CSA-binding sequence substantially as shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11, or at least 95% identical thereto, or the corresponding portion of PfEMP1 from a strain of Plasmodium, substantially in isolation from sequences naturally occurring adjacent thereto in the PfEMP1 protein.

48. The polypeptide of claim 47 wherein the SEQ ID NO is SEQ ID NO: 1.

49. The polypeptide of claim 47 wherein the SEQ ID NO is SEQ ID NO: 2.

50. The polypeptide of claim 47 wherein the SEQ ID NO is SEQ ID NO: 3.

51. The polypeptide of claim 47 wherein the SEQ ID NO is SEQ ID NO: 4.

52. The polypeptide of claim 47 wherein the SEQ ID NO is SEQ ID NO: 5.

53. The polypeptide of claim 47 wherein the SEQ ID NO is SEQ ID NO: 6.

54. The polypeptide of claim 47 wherein the SEQ ID NO is SEQ ID NO: 7.

55. The polypeptide of claim 47 wherein the SEQ ID NO is SEQ ID NO: 8.

56. The polypeptide of claim 47 wherein the SEQ ID NO is SEQ ID NO: 9.

57. The polypeptide of claim 47 wherein the SEQ ID NO is SEQ ID NO: 10.

58. The polypeptide of claim 47 wherein the SEQ ID NO is SEQ ID NO: 11.

59. An isolated nucleotide sequence encoding a polypeptide comprising a CSA-binding sequence substantially as shown in SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11, or at least 95% identical thereto, or the corresponding portion of PfEMP1 from a strain of Plasmodium, substantially in isolation from sequences naturally occurring adjacent thereto in the PfEMP1 protein.

60. The nucleotide sequence of claim 59 wherein the SEQ ID NO is SEQ ID NO: 1.

61. The nucleotide sequence of claim 59 wherein the SEQ ID NO is SEQ ID NO: 2.

62. The nucleotide sequence of claim 59 wherein the SEQ ID NO is SEQ ID NO: 3.

63. The nucleotide sequence of claim 59 wherein the SEQ ID NO is SEQ ID NO: 4.

64. The nucleotide sequence of claim 59 wherein the SEQ ID NO is SEQ ID NO: 5.

65. The nucleotide sequence of claim 59 wherein the SEQ ID NO is SEQ ID NO: 6.

66. The nucleotide sequence of claim 59 wherein the SEQ ID NO is SEQ ID NO: 7.

67. The nucleotide sequence of claim 59 wherein the SEQ ID NO is SEQ ID NO: 8.

68. The nucleotide sequence of claim 59 wherein the SEQ ID NO is SEQ ID NO: 9.

69. The nucleotide sequence of claim 59 wherein the SEQ ID NO is SEQ ID NO: 10.

70. The nucleotide sequence of claim 59 wherein the SEQ ID NO is SEQ ID NO: 11.

71. A vector, comprising the nucleotide sequence of claim 59.

72. The vector according to claim 71, which when inserted into a suitable host cell allows for the expression of a polypeptide comprising a CSA-binding sequence substantially as shown in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11, or at least 95% identical thereto, or the corresponding portion of PfEMP1 from a strain of Plasmodium, substantially in isolation from sequences naturally occurring adjacent thereto in the PfEMP1 protein.

73. The vector according to claim 72, wherein said polypeptide is expressed as a fusion protein.

74. A method of making a polypeptide comprising a CSA-binding sequence substantially as shown in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11, or at least 95% identical thereto, or the corresponding portion of PfEMP1 from a strain of Plasmodium, substantially in isolation from sequences naturally occurring adjacent thereto in the PfEMP1 protein, said method comprising the steps of introducing the vector of claim 71 into a suitable host cell; growing said host cell; and isolating the polypeptide so produced.

75. A host cell transformed with a vector according to claim 71.

76. A vaccine suitable for use in the prevention and/or treatment of malaria due to Plasmodium, said vaccine comprising at least one polypeptide comprising a CSA-binding sequence substantially as shown in any one of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, or SEQ ID NO: 11, or at least 95% identical thereto, or the corresponding portion of PfEMP1 from a strain of Plasmodium, substantially in isolation from sequences naturally occurring adjacent thereto in the PfEMP1 protein, said vaccine further comprising a physiologically acceptable carrier.

77. The vaccine according to claim 76, wherein said polypeptide is present as a fusion protein.

78. A method of preventing and/or treating a human body for malaria especially in pregnancy due to Plasmodium, comprising administering an effective amount of a vaccine according to claim 76.

79. An isolated polypeptide comprising a CSA-binding sequence substantially as shown in SEQ ID NO: 12, SEQ ID NO: 13, SEQ ID NO: 14, or SEQ ID NO: 15, or at least 95% identical thereto, or a CSA-binding fragment thereof, substantially in isolation from sequences naturally occurring adjacent thereto in the PfEMP1 protein.

80. The polypeptide of claim 79 wherein the SEQ ID NO is SEQ ID NO: 12.

81. The polypeptide of claim 79 wherein the SEQ ID NO is SEQ ID NO: 13.

82. The polypeptide of claim 79 wherein the SEQ ID NO is SEQ ID NO: 14.

83. The polypeptide of claim 79 wherein the SEQ ID NO is SEQ ID NO: 15.

84. An isolated nucleotide sequence encoding a polypeptide comprising a CSA-binding sequence substantially as shown in SEQ ID NO: 12, SEQ ID NO: 13, SEQ ID NO: 14, or SEQ ID NO: 15, or at least 95% identical thereto, or a CSA-binding fragment thereof, substantially in isolation from sequences naturally occurring adjacent thereto in the PfEMP1 protein.

85. The nucleotide sequence of claim 84 wherein the SEQ ID NO is SEQ ID NO: 12.

86. The nucleotide sequence of claim 84 wherein the SEQ ID NO is SEQ ID NO: 13.

87. The nucleotide sequence of claim 84 wherein the SEQ ID NO is SEQ ID NO: 14

88. The nucleotide sequence of claim 84 wherein the SEQ ID NO is SEQ ID NO: 15.

89. A vector, comprising the nucleotide sequence of claim 84.

90. The vector according to claim 89, which when inserted into a suitable host cell allows for the expression of a polypeptide comprising a CSA-binding sequence substantially as shown in any one of SEQ ID NO: 12, SEQ ID NO: 13, SEQ ID NO: 14, or SEQ ID NO: 15, or at least 95% identical thereto, or a CSA-binding fragment thereof, substantially in isolation from sequences naturally occurring adjacent thereto in the PfEMP1 protein.

91. The vector according to claim 90, wherein said polypeptide is expressed as a fusion protein.

92. A method of making a polypeptide comprising a CSA-binding sequence substantially as shown in any one of SEQ ID NO: 12, SEQ ID NO: 13, SEQ ID NO: 14, or SEQ ID NO: 15, or at least 95% identical thereto, or a CSA-binding fragment thereof, substantially in isolation from sequences naturally occurring adjacent thereto in the PfEMP1 protein, said method comprising the steps of introducing the vector of claim 89 into a suitable host cell; growing said host cell; and isolating the polypeptide so produced.

93. A host cell transformed with a vector according to claim 89.

94. A vaccine suitable for use in the prevention and/or treatment of malaria due to Plasmodium, said vaccine comprising at least one polypeptide comprising a CSA-binding sequence substantially as shown in any one of SEQ ID NO: 12, SEQ ID NO: 13, SEQ ID NO: 14, or SEQ ID NO: 15, or at least 95% identical thereto, or a CSA-binding fragment thereof, substantially in isolation from sequences naturally occurring adjacent thereto in the PfEMP1 protein, said vaccine further comprising a physiologically acceptable carrier.

95. The vaccine according to claim 94, wherein said polypeptide is present as a fusion protein.

96. A method of preventing and/or treating a human body for malaria especially in pregnancy due to Plasmodium, comprising administering an effective amount of a vaccine according to claim 94.
